(19) United States
(12) Patent Application Publication
(10) Pub. No.: US 2026/0041813 A1
(43) Pub. Date: Feb. 12, 2026

(54) THREE-DIMENSIONAL NETWORK AQUEOUS GEL

(71) Applicant: Nai-Wen MEI, New Taipei City (TW)

(72) Inventors: Nai-Wen MEI, Taipei City (TW); Hui-Ching MEI, New Taipei City (TW)

(21) Appl. No.: 19/309,634

(22) Filed: Aug. 26, 2025

Related U.S. Application Data
(63) Continuation-in-part of application No. 18/095,538, filed on Jan. 11, 2023.

(30) Foreign Application Priority Data
Jan. 11, 2022 (TW) 111101154

(51) Int. Cl. A61L 26/00

(52) U.S. Cl. CPC A61L 26/008; A61L 26/0014; A61L 26/0023; A61L 26/0095

(57) ABSTRACT

A three-dimensional network aqueous gel is composed of a water-soluble polymer including sodium carboxymethyl cellulose, sodium alginate, and polyvinylpyrrolidone. The water-soluble polymer and a solvent are crosslinked to form a three-dimensional network structure. The water-soluble polymer comprises 10-30 wt % of sodium alginate, 10-30 wt % of polyvinylpyrrolidone, and 10-40 wt % of sodium carboxymethyl cellulose. The solvent is selected from high-oxygen water and has a dissolved oxygen concentration of 25 to 35 mg/L. The three-dimensional network structure includes a plurality of gel pores formed therein. The plurality of gel pores have a diameter of 16-18 μm and form a continuous pore structure that provides an oxygen-permeable environment with a high oxygen content. When applied to a wound, the three-dimensional network structure quickly forms a thin film that increases mechanical tensile properties, promotes wound healing and tissue regeneration, and has stability, transparency, and moisturizing ability.

### CROSS REFERENCE TO RELATED APPLICATIONS

This is a continuation-in-part of co-pending U.S. patent application Ser. No. 18/095,538 filed on Jan. 11, 2023.

### BACKGROUND OF THE INVENTION

### Technical Field

The present invention relates to a three-dimensional network aqueous gel, and more particularly to a three-dimensional network aqueous gel that provides an oxygen-permeable environment with a high oxygen content to increase mechanical tensile properties, promote wound healing and tissue regeneration, and have stability, transparency, and moisturizing ability as well as speed up healing of wounds of all sorts and prevent abnormal healing of wounds.

### Description of Related Art

Skin is the largest organ of a human body and has functions of protecting against invasion of external bacteria, regulating balance of body liquid, and sensing external excitations. When a wound appears on the skin, the skin is no longer capable of the function of protecting. The wound, if not properly handled, may lead to infection or inflammation. Thus, the wound must be handled in a correct way to avoid wound infection. It is a common practice to use a dressing having functions of hemostasis, protection, and infection prevention to cover the wound. Further, the addressing can increase the rate of skin repair and regeneration, and thus, the solution for accelerating wound healing is also a vital issue.

The dressings that are contemporarily used to cover wounds are classified as dry dressings and wet dressings. The dry dressings include gauze, and the wet dressings include film dressings, hydrophilic dressings, hydrophilic fibrous dressings, foam dressings, antimicrobial dressings, negative-pressure wound dressings, advanced therapy dressings, and active dressings. The dry dressings have the following defects: poor healing environment, easy localized dehydration of wound site, formation of scab, and wound pain caused by scabs, and consequently, loss of bioactivity, slow healing rate, and frequent replacement of dressings for fast leakage. Further, the dressing may get adhered to newly growing granulation tissue so that the wound would be damaged in replacing the dressings, and in addition, there is no isolative barrier from the outside, making the chance of cross infection increased.

Furthermore, although traditional wet dressings have good hydrophilicity and film-forming ability, their structure is loose, their transparency and mechanical strength are insufficient, and they cannot effectively provide an oxygen-containing microenvironment. To date, there is still a lack of a gel material that combines oxygen-containing ability, good mechanical tensile properties, stability, transparency, and moisturizing ability.

### SUMMARY OF THE INVENTION

The primary objective of the present invention is to provide a three-dimensional network aqueous gel that provides an oxygen-permeable environment with a high oxygen content to increase mechanical tensile properties, promote wound healing and tissue regeneration, and have stability, transparency, and moisturizing ability as well as speed up healing of wounds of all sorts and prevent abnormal healing of wounds.

To achieve the above objective, the present invention provides a three-dimensional network aqueous gel composed of a water-soluble polymer. The water-soluble polymer includes sodium carboxymethyl cellulose, sodium alginate, and polyvinylpyrrolidone. The water-soluble polymer and a solvent are crosslinked to form a three-dimensional network structure. The water-soluble polymer includes 10-30 wt % of sodium alginate, 10-30 wt % of polyvinylpyrrolidone, and 10-40 wt % of sodium carboxymethyl cellulose, calculated based on 100 wt % of the total weight percentage of the water-soluble polymer. The solvent is selected from high-oxygen water and has a dissolved oxygen concentration of 25 to 35 mg/L. The three-dimensional network structure includes a plurality of gel pores formed therein. The plurality of gel pores have a diameter of 16-18 μm and form a continuous pore structure that provides an oxygen-permeable environment with a high oxygen content. When applied to a wound, the three-dimensional network structure quickly forms a thin film that increases mechanical tensile properties, promotes wound healing and tissue regeneration, and has stability, transparency, and moisturizing ability.

The present invention has at least the following effects:

1. Oxygen supply from high-oxygen water: The dissolved oxygen contained in the high-oxygen water is stably present in the water-soluble polymer in a physically dissolved state. When applied to the skin, oral cavity, or mucosal surfaces, the gel contacts the body temperature or physiological fluids, causing a gradient difference in dissolved oxygen concentration in the microenvironment. This promotes the release of oxygen from the gel into the tissue surface, helping to improve the oxygen uptake ability of cells, promote collagen synthesis, activate cells, and promote angiogenesis and repair.

2. Stable release mechanism of the three-dimensional network structure: The pores of the three-dimensional network structure can stably control the diffusion and release of oxygen, water, and nutrients, providing a humid or wet but not overly wet healing environment, extending the duration of the oxygen-containing state and preventing instantaneous depletion.

3. Moisturizing and film-forming principle of hydrophilic polymers: The sodium carboxymethyl cellulose, sodium alginate, and polyvinylpyrrolidone used are all highly hydrophilic and adhesive biomaterials. They can form a soft aqueous film on the surface of damaged tissue, reducing water evaporation and cracking of the wound, blocking external contamination and pathogenic microorganisms, and enhancing the absorption of local drugs or nutrients.

### BRIEF DESCRIPTION OF THE DRAWINGS

The present disclosure can be more fully understood by reading the following detailed description of the embodiment, with reference made to the accompanying drawing as follows:

FIG. 1 is a schematic view illustrating formation of a three-dimensional network aqueous gel according to the present invention;

FIG. 2 is a scanning electronic microscope (SEM) photograph of the three-dimensional network aqueous gel according to the present invention;

FIG. 3 is a flow chart showing a manufacturing method for formation of the three-dimensional network aqueous gel according to the present invention;

FIG. 4A shows a picture of an affected part of a bedsore patient taken before being applied with the three-dimensional network aqueous gel according to the present invention;

FIG. 4B shows a picture of the affected part of the bedsore patient taken after having been applied with the three-dimensional network aqueous gel according to the present invention for two (2) days;

FIG. 4C shows a picture of the affected part of the bedsore patient taken after having been applied with the three-dimensional network aqueous gel according to the present invention for seven (7) days;

FIG. 5A shows a picture of a diabetes foot taken before being applied with the three-dimensional network aqueous gel according to the present invention;

FIG. 5B shows a picture of the diabetes foot taken after having been applied with the three-dimensional network aqueous gel according to the present invention for two (2) days;

FIG. 5C shows a picture of the diabetes foot taken after having been applied with the three-dimensional network aqueous gel according to the present invention for seven (7) days;

FIG. 6 shows a SEM image of the three-dimensional network aqueous gel of the present invention; and

FIG. 7 shows a diagram of the blood oxygen changes in mice after feeding.

### DETAILED DESCRIPTION OF THE PREFERRED EMBODIMENTS

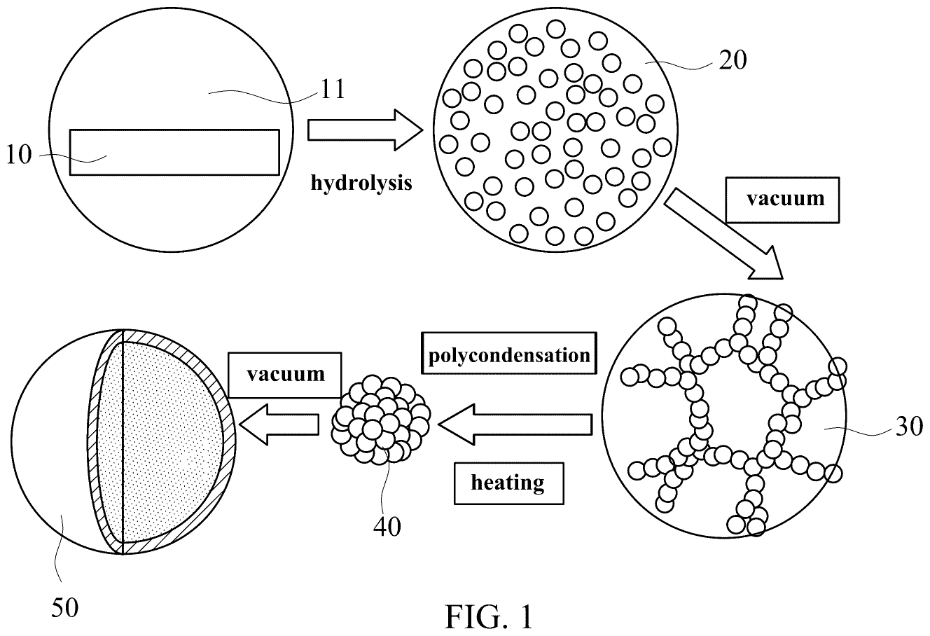

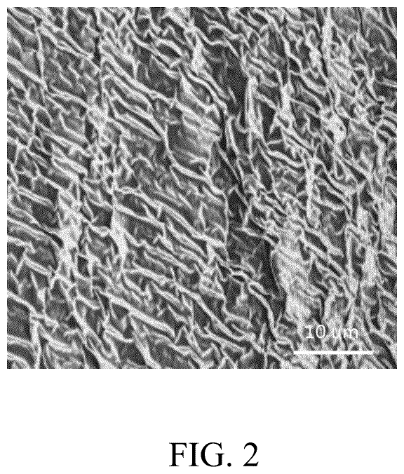

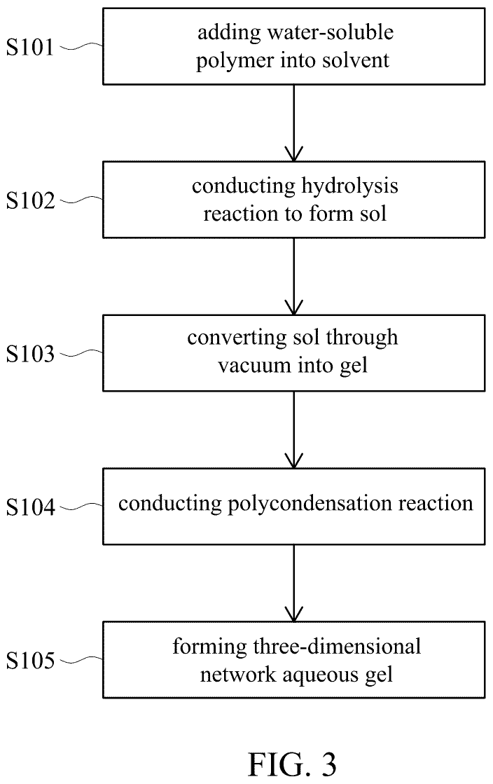

Referring to FIGS. 1-3, the present invention provides a three-dimensional network aqueous gel, which is composed of a water-soluble polymer 10. The water-soluble polymer 10 includes sodium carboxymethyl cellulose, sodium alginate, and polyvinylpyrrolidone. The water-soluble polymer 10 and a solvent 11 are crosslinked to form a three-dimensional network structure.

The water-soluble polymer 10 includes 10-30 wt % of sodium alginate, 10-30 wt % of polyvinylpyrrolidone, and 10-40 wt % of sodium carboxymethyl cellulose, calculated based on 100 wt % of the total weight percentage of the water-soluble polymer 10.

In particular, the solvent 11 is selected from high-oxygen water and has a dissolved oxygen concentration of 25 to 35 mg/L. The three-dimensional network structure includes a plurality of gel pores formed therein. The plurality of gel pores have a diameter of 16-18 μm and form a continuous pore structure that provides an oxygen-permeable environment with a high oxygen content; when applied to a wound, the three-dimensional network structure quickly forms a thin film that increases mechanical tensile properties, promotes wound healing and tissue regeneration, and has stability, transparency, and moisturizing ability.

In particular, the high-oxygen water is prepared by introducing pure oxygen into purified water.

In particular, the tensile strength of the gel is greater than 0.5 MPa.

In particular, a weight retention rate of the gel is greater than 90% after 72 hours in a humid or wet environment with 85% relative humidity and a temperature of 37° C.

In particular, an optical transmittance of the gel is greater than 90% in the visible light range of 500-700 nm.

### Embodiment of Manufacturing Method

The present invention provides a manufacturing method of a three-dimensional network aqueous gel. The method comprises: Step S101: adding a water-soluble polymer in an organic solvent and uniformly mixing together to form a homogeneous solution, wherein the water-soluble polymer 10 includes 10-30 wt % of sodium alginate, 10-30 wt % of polyvinylpyrrolidone, and 10-40 wt % of sodium carboxymethyl cellulose, calculated 10-30 wt % of sodium alginate, 10-30 wt % polyvinylpyrrolidone, and 10-40 wt % sodium carboxymethyl cellulose, calculated based on the total weight percentage of 100 wt % of the water-soluble polymer. Step S102: subjecting the water-soluble polymer to a hydrolysis reaction to generate nanometer-scale water-soluble polymer particles and form a sol, the sol being a colloidal system having liquid characteristics having dispersed particles being solid or macromolecules, the dispersed particles having a size between 1-100 nm. Step S103: under a pressure of 25-760 torr, subjecting the sol to vacuum conversion into a gel, the gel being a colloidal system having solid characteristics, the dispersed substance forming a continuous network framework, framework gaps being filled with liquid or gas, the content of a dispersion phase in the gel being extremely low, generally between 1%-3%. Step S104: subjecting the gel to a polycondensation reaction and controlling a heating temperature between 30-70 degrees and a pressure between 50-70 millimeters of mercury, so as to induce interconnection between the nanometer-scale water-soluble polymer particles to form a three-dimensional network structure, wherein, specifically, the three-dimensional network structure provides the gel with an extremely high specific surface area. Step S105: subjecting the three-dimensional network structure to vacuum to form a three-dimensional network aqueous gel, wherein the three-dimensional network structure comprises a plurality of gel pores formed therein, and the gel pores have a diameter between 16-18 μm.

### Swelling Degree Test

By controlling pressure and temperature, the structure of the three-dimensional network aqueous gel can be changed to regulate the degree of swelling of the three-dimensional network aqueous gel. The higher the temperature and pressure are, the smaller the diameter of the gel pores and the smaller the degree of swelling. The degree of swelling is calculated by means of the following formula: {(weight of three-dimensional network aqueous gel after swelling-weight of three-dimensional network aqueous gel before swelling)/weight of three-dimensional network aqueous gel before swelling χ 100}. In the swelling degree test, the water-soluble polymer of the three-dimensional network aqueous gel comprises 30 wt % of sodium alginate, 30 wt % of polyvinylpyrrolidone, and 40 wt % of sodium carboxymethyl cellulose, based on 100 wt % of the total weight percentage of the water-soluble polymer, and the result of the change of the gel pore and the degree of swelling of the three-dimensional network aqueous gel by means of temperature and pressure is shown in Table 1.

TABLE 1

| | | gel pore | degree of |
|---|---|---|---|
| temperature | pressure | diameter | swelling |
| (° C.) | (mmHg) | (μm) | (%) |
| 30 | 50 | 17.8 | 44.03% |
| 35 | 50 | 17.8 | 45.07% |
| 40 | 50 | 17.3 | 42.05% |
| 45 | 50 | 16.9 | 40.12% |
| 50 | 70 | 16.8 | 40.01% |
| 55 | 70 | 16.5 | 38.57% |
| 60 | 70 | 16.6 | 37.23% |
| 65 | 70 | 16.3 | 36.11% |
| 70 | 70 | 16.2 | 35.74% |

### Controlled Release Test

The water-soluble polymer of the three-dimensional network aqueous gel comprises 30 wt % of sodium alginate, 30 wt % of polyvinylpyrrolidone, and 40 wt % of sodium carboxymethyl cellulose, based on 100 wt % of the total weight percentage of the water-soluble polymer, and the controlled release time can be achieved by adjusting the three-dimensional network structure to provide an effect of controlled release. The controlled release time is estimated by using a transdermal absorption test apparatus, and pig skin is used as an artificial skin. The result is shown in the following Table 2.

TABLE 2

| | | gel pore | |
|---|---|---|---|
| temperature | pressure | diameter | controlled release |
| (° C.) | (mmHg) | (μm) | time (days) |
| 30 | 50 | 17.8 | 7 |
| 35 | 50 | 17.8 | 7 |
| 40 | 50 | 17.3 | 7.5 |
| 45 | 50 | 16.9 | 7 |
| 50 | 70 | 16.8 | 8 |
| 55 | 70 | 16.5 | 11 |
| 60 | 70 | 16.6 | 10.5 |
| 65 | 70 | 16.3 | 11 |
| 70 | 70 | 16.2 | 12 |

### Wound Healing Test

### Test Animal

The test uses male New Zealand rabbits of 8 weeks old, having a body weight of approximately 2000-2500 g. All the test animals are raised in an animal room having independent air conditioning with the room temperature being kept at 22° C. and relative humidity being kept at 45%, water and feed being sufficiently supplied. Before the test, the animals are given four weeks for adaption to the environment. Feeding environment, handling and all test procedures are in full compliance with “Guide for the Care and Use of Laboratory Animals” issued by (National Institutes of Health (NIH)).

### Formation of Skin Wound

The back part of the New Zealand rabbit is shaved, and sterilized with iodine tincture and 70% alcohol, and then, a skin wound of an area around 2 cm×2 cm is made on the back of the New Zealand rabbit by cutting with a surgical knife.

### Compositional Ingredients of Water-Soluble Polymer

TEST GROUP 1: The water-soluble polymer comprises, in the total weight percentage thereof, 25 wt % of sodium alginate, 25 wt % of polyvinylpyrrolidone, and 50 wt % of sodium carboxymethyl cellulose, calculated on the basis of 100 wt % of the total weight percentage of the water-soluble polymer.

TEST GROUP 2: The water-soluble polymer comprises, in the total weight percentage thereof, 30 wt % of sodium alginate, 30 wt % of polyvinylpyrrolidone, and 40 wt % of sodium carboxymethyl cellulose, calculated on the basis of 100 wt % of the total weight percentage of the water-soluble polymer.

COMPARISON GROUP 1: The water-soluble polymer comprises, in the total weight percentage thereof, 25 wt % of sodium alginate, 25 wt % of polyvinylpyrrolidone, and 50 wt % of sodium carboxymethyl cellulose, calculated on the basis of 100 wt % of the total weight percentage of the water-soluble polymer.

COMPARISON GROUP 2: The water-soluble polymer comprises, in the total weight percentage thereof, 30 wt % of sodium alginate, 30 wt % of polyvinylpyrrolidone, and 40 wt % of sodium carboxymethyl cellulose, calculated on the basis of 100 wt % of the total weight percentage of the water-soluble polymer.

### Application of Three-Dimensional Network Aqueous Gel

New Zealand rabbits are randomly divided into two test groups and two comparison groups, wherein the New Zealand rabbits of each of the groups are formed with a skin wound as described above, and then, the skin wounds of the New Zealand rabbits of TEST GROUPS 1 and 2 are applied with the three-dimensional network aqueous gels according to the above TEST GROUPS 1 and 2, and the wounds of the animals are covered with a polyurethane (PU) waterproof film to keep humid. The New Zealand rabbits of COMPARISON GROUPS are formed with a skin wound as described above, and then, the skin wounds of the New Zealand rabbits of COMPARISON GROPUS 1 and 2 are applied with the three-dimensional network aqueous gels according to the above COMPARISON GROUPS 1 and 2. The test is conducted for 14 days in total, and the wound areas of the New Zealand rabbits of each group are measured at the 2nd, 7th, and 14th days after the application of the dressing. The result is shown in the following Table 3.

TABLE 3

| | 2nd day after | 7th day after | 14th day after |
|---|---|---|---|
| | application | application | application |
| TEST GROUP 1 | 3.74 cm² | 3.35 cm² | 3.01 cm² |
| TEST GROUP 2 | 3.54 cm² | 3.09 cm² | 2.95 cm² |
| COMPARISON GROUP 1 | 3.96 cm² | 3.74 cm² | 3.61 cm² |
| COMPARISON GROUP 2 | 3.67 cm² | 3.44 cm² | 3.35 cm² |

A difference between TEST GROUP 1 and TEST GROUP 2 is the total weight percentage of the water-soluble polymer. The content of sodium carboxymethyl cellulose of TEST GROUP 2 is greater than that of TEST GROUP 1, and it is known from the above Table that a high content of sodium carboxymethyl cellulose increases the wound closure rate. Next, TEST GROUP 1 and TEST GROUP 2 cover a water-resistant film of polyurethane to keep the wound wet, while COMPARISON GROUP 1 and COMPARISON GROUP 2 do not apply a water-resistant film of polyurethane to cover the wounds, and it is known from the above Table that the healing rate can be much faster if the wounds are kept in a humid or wet environment. In summary of the above, wet healing of a wound provides the following advantage. Firstly, it is advantageous for dissolution of necrotic tissues and fibrins, in a humid or wet environment, tissue plasmin contained in wound exudate may prompt the dissolution and absorption of the necrotic tissue. Further, it keeps the wound site at a fixed temperature, accelerate division of cells, prompt wound healing, develop localized wetting and reduce formation of scabs, prevent mechanical damage to newly growing granulation tissues, reduce damages and pains for change of dressing, protect nerve endings at the wound to reduce pain. Further, in the closed wet-keeping environment, the dressing forms a barrier to reduce the chance of infection, and the slightly acidic environment in the closed condition could suppress growth of bacteria and help proliferation and functioning of white blood cells.

The three-dimensional network aqueous gel manufactured with the high-oxygen-content aqueous gel manufacturing method according to the present invention features containing sodium carboxymethyl cellulose, polyvinylpyrrolidone, and sodium alginate and may form a colloidal body having high adhesion power, and can be made as a network polymer colloid that contains a great amount of water and have adhering property and excellent water absorbability. The colloid, when put in contact with a body surface, may induce repeated hydration reaction and exhibiting dual functions of supplying water toward the surface and absorbing exudate, by which bleeding and loss of body fluid can be controlled. Hydrophilic groups of sodium carboxymethyl cellulose, after absorbing water, becomes a gel form adhering to the wound site of blood vessel and swelling to form a gel layer to achieve wound hemostasis. Next, the high-oxygen-content aqueous gel forms a protective layer on the surface of a wound, which is colorless and clear, having a high moisture content, so as to keep the wound humid, prevent rubbing and irritating of the wound, not damaging the newly growing granulation tissues, and reducing secondary damage. Since sodium carboxymethyl cellulose contains acidic carboxyl group that is combinable with Fe²⁺ of hemoglobin to form a brown adhesive colloid block that achieves closure of endings of capillary vessels for stop bleeding. Further, the colloidal body also shows an effect of adhering and aggregating for platelets to thereby speed up blood clotting.

### Human Body Test

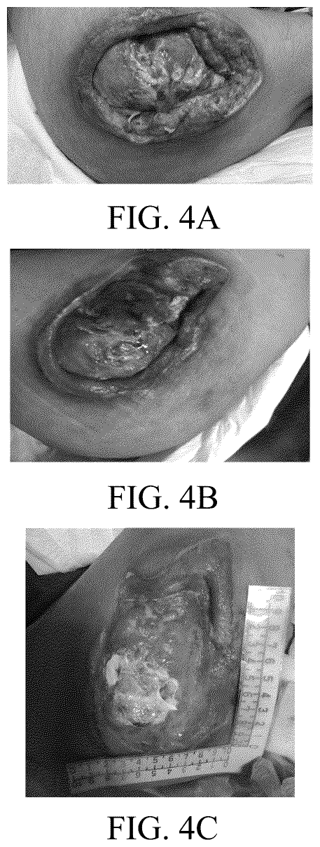

Referring to FIGS. 4A-4C, FIG. 4A shows a picture of an affected part of a bedsore patient taken before being applied with the three-dimensional network aqueous gel according to the present invention; FIG. 4B shows a picture of the affected part of the bedsore patient taken after having been applied with the three-dimensional network aqueous gel according to the present invention for two (2) days; and FIG. 4C shows a picture of the affected part of the bedsore patient taken after having been applied with the three-dimensional network aqueous gel according to the present invention for seven (7) days.

As shown in FIGS. 4A-4C, SUBJECT 1 of EMBODIMENT 1 is a bedsore patient. The wound of SUBJECT 1 is observable for fat tissues, and gives off odors, where granulation tissues, and edge curling, carrions, or scabs of the wound are observable, but fascia, muscle, tendon, ligament, cartilage, and bone are not observable. Application of the three-dimensional network aqueous gel according to the present invention is made to the affected part of the patient and observing the healing condition of the wound for 0 to 7 days. In the total weight percentage, the water-soluble polymer comprises 25 wt % of sodium alginate, 25 wt % of polyvinylpyrrolidone, and 50 wt % of sodium carboxymethyl cellulose, calculated on the basis of 100 wt % of the total weight percentage of the water-soluble polymer. After two days, blackening of skin has been improved, and the fat tissues in the wound become smaller after application for 7 days.

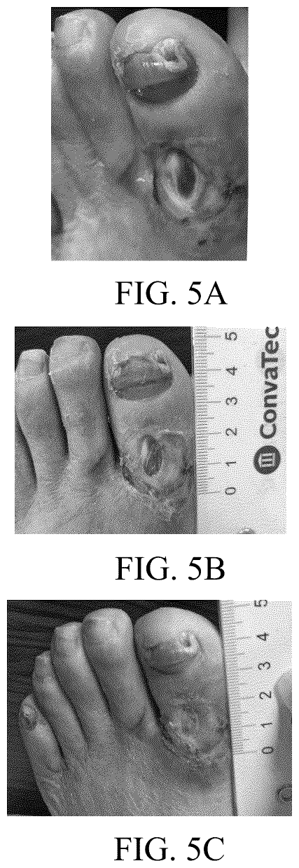

Referring to FIGS. 5A-5C, FIG. 5A shows a picture of a diabetes foot taken before being applied with the three-dimensional network aqueous gel according to the present invention; FIG. 5B shows a picture of the diabetes foot taken after having been applied with the three-dimensional network aqueous gel according to the present invention for two (2) days; and FIG. 5C shows a picture of the diabetes foot taken after having been applied with the three-dimensional network aqueous gel according to the present invention for seven (7) days.

As shown in FIGS. 5A-5C, SUBJECT 2 of EMBODIMENT 2 is a diabetes patient, and the foot tendon and ligament tissues have ulcerated and suppurative discharges and tissue necrosis have increased. After application of the three-dimensional network aqueous gel according to the present invention is made to the affected part of the patient and making observation for the healing condition of the wound for 0 to 7 days. In the total weight percentage, the water-soluble polymer comprises 25 wt % of sodium alginate, 25 wt % of polyvinylpyrrolidone, and 50 wt % of sodium carboxymethyl cellulose, calculated on the basis of 100 wt % of the total weight percentage of the water-soluble polymer. After two days, the ulcer improved, and the wound is gradually healing after 7 days and the range of the wound shrinks.

In summary, the present invention provides a three-dimensional network aqueous gel, and a manufacturing method thereof. The three-dimensional network aqueous gel, upon application, forms a layer of extremely thin and invisible film on the surface of skin within 1 to 3 minutes. The film so formed, and the three-dimensional network structure contained in the film, can more effectively protect API (Active Pharmaceutical Ingredient) and achieve an effect of controlled release. The controlled release time can be varied to reach, maximally, up to 12 days by adjusting the three-dimensional network structure. Further, the three-dimensional network aqueous gel has effects of antiinflammation, smoothing wound, and preventing abnormal healing of wound, so as to achieve the purpose of speeding up wound healing and reducing pain of wound dressing change.

### Mechanical Strength Test

Experimental method: The tensile strength was tested according to ASTM D882 test method. Strip-shaped samples (60 mm×10 mm×2 mm) of the prepared gel were subjected to tension on a tensile testing machine (Instron 3345) at a speed of 50 mm/min until they broke. The maximum stress was recorded.

### Experimental Results:

| Test Item | Average Value ± SD |
|---|---|
| Tensile Strength | 0.58 ± 0.04 MPa |

The gel of the present invention exhibits excellent mechanical stability, making it suitable for applications that require adhesion to oral or skin surfaces.

### Moisturizing Ability Test

Experimental method: Gel samples (20 mm×20 mm×3 mm) were prepared, and their initial weight was recorded as Wₒ. The samples were placed in a constant temperature and humidity chamber at 37° C. and 85% relative humidity. The weight Wₙ was measured once every 24 hours (weighed immediately to avoid volatilization or moisture loss). The weight retention rate was calculated as: Moisturizing Rate (%)=Wₙ/Wₒ×100.

### Experimental Results:

| Time (hr) | Moisturizing Rate (%) |
|---|---|
| 24 | 98.3% |
| 48 | 94.8% |
| 72 | 90.5% |

The gel of the present invention can maintain more than 90% of its water content within three days, proving that it has good moisturizing and sustained-release ability.

### Optical Transparency Test

Experimental method: A UV-Vis spectrophotometer (Shimadzu UV-2600) was used. The test sample was a homogeneous gel with a thickness of 2 mm, placed in a quartz cuvette. The wavelength range was 500-700 nm, and the scan step was 1 nm. The results are expressed as average transmittance.

### Experimental Results:

| Wavelength Range | Transmittance (Average ± SD) |
|---|---|
| 500-700 nm | 91.7% ± 1.2% |

The gel of the present invention has high transparency, which is beneficial for dressing visibility or biomedical optical observation.

### Oxygen Supply Capacity

### Experimental Basis and Application Examples:

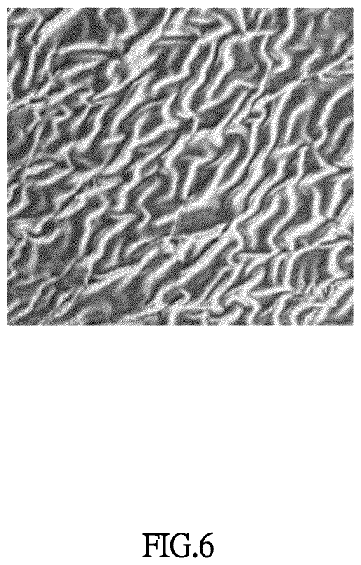

As shown in FIG. 6, the SEM image shows that the gel has pores with a diameter of 16-18 μm, which are gas-permeable channels. The oxygen release test shows that the dissolved oxygen concentration is still >8 mg/L after 24 hours of application. It is suitable for local application on oral ulcers, burns, and mucosal injuries to increase the oxygen content of the wound microenvironment, which is beneficial for promoting fibroblast proliferation and angiogenesis.

### Mouse Blood Po₂ Increase Test

### Experimental Method:

The test animals were BALB/c mice. The test method was to gavage a high-oxygen gel sample, and the control group was a anaerobic gel. Blood (0.2 mL) was collected from the tail vein every 30 minutes, and the pO₂ (mmHg) was immediately measured using an i-STAT Portable Blood Analyzer.

### Experimental Results:

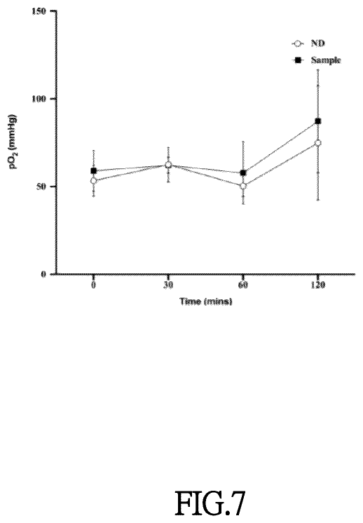

As shown in FIG. 7.

| Time (min) | Control Group (ND) | High-Oxygen Gel Group |
|---|---|---|
| 0 | 52.1 ± 4.2 | 59.3 ± 5.1 |
| 60 | 50.9 ± 4.1 | 57.7 ± 5.2 |
| 120 | 72.0 ± 6.8 | 83.5 ± 5.6 |

The results show that the high-oxygen gel group significantly increased blood pO₂, indicating a systemic oxygen supply ability.

What is claimed is:

1. A three-dimensional network aqueous gel composed of a water-soluble polymer, the water-soluble polymer comprising sodium carboxymethyl cellulose, sodium alginate, and polyvinylpyrrolidone, wherein the water-soluble polymer and a solvent are crosslinked to form a three-dimensional network structure;
wherein the water-soluble polymer comprises 10-30 wt % of sodium alginate, 10-30 wt % of polyvinylpyrrolidone, and 10-40 wt % of sodium carboxymethyl cellulose, calculated based on 100 wt % of the total weight percentage of the water-soluble polymer;
wherein the solvent is selected from high-oxygen water and has a dissolved oxygen concentration of 25 to 35 mg/L; the three-dimensional network structure comprises a plurality of gel pores formed therein; the plurality of gel pores have a diameter of 16-18 μm and form a continuous pore structure that provides an oxygen-permeable environment with a high oxygen content; when applied to a wound, the three-dimensional network structure quickly forms a thin film that increases mechanical tensile properties, promotes wound healing and tissue regeneration, and has stability, transparency, and moisturizing ability.

2. The three-dimensional network aqueous gel as claimed in claim 1, wherein the high-oxygen water is prepared by introducing pure oxygen into purified water.

3. The three-dimensional network aqueous gel as claimed in claim 1, wherein the tensile strength of the gel is greater than 0.5 MPa.

4. The three-dimensional network aqueous gel as claimed in claim 1, wherein a weight retention rate of the gel is greater than 90% after 72 hours in a humid or wet environment with 85% relative humidity and a temperature of 37° C.

5. The three-dimensional network aqueous gel as claimed in claim 1, wherein an optical transmittance of the gel is greater than 90% in the visible light range of 500-700 nm.
